(19) United States
(12) Patent Application Publication
(10) Pub. No.: US 2012/0330006 A1
(43) Pub. Date: Dec. 27, 2012

(54) METHOD OF MANUFACTURING EPSILON-CAPROLACTAM

(76) Inventor: Hideto Nagami, Niihama-shi (JP)

(21) Appl. No.: 13/581,777

(22) PCT Filed: Mar. 1, 2011
(86) PCT No.: PCT/JP2011/001176

§ 371 Date: Aug. 29, 2012

(30) Foreign Application Priority Data
Mar. 2, 2010 (JP) 2010-045649

(51) Int. Cl. C07D 201/16

(52) U.S. Cl. 540/540

(57) ABSTRACT

An epsilon-caprolactam manufacturing method capable of manufacturing in good yield, high-quality epsilon-caprolactam containing less impurity has an epsilon-caprolactam purification step A of obtaining purified epsilon-caprolactam from raw epsilon-caprolactam by applying a drop crystallization method, a first-stage epsilon-caprolactam recovery step B of obtaining first recovered epsilon-caprolactam and a first recovered mother liquor by applying an evaporative crystallization method to a crystallization mother liquor obtained in the epsilon-caprolactam purification step A, and a second-stage epsilon-caprolactam recovery step C of obtaining second recovered purified epsilon-caprolactam by applying a melt crystallization method to the first recovered mother liquor, first recovered epsilon-caprolactam being recovered as a raw material for the epsilon-caprolactam purification step A and second recovered purified epsilon-caprolactam being recovered as a raw material for the epsilon-caprolactam purification step A and/or the first-stage epsilon-caprolactam recovery step B.

### TECHNICAL FIELD

The present invention relates to a method of manufacturing epsilon-caprolactam, and particularly to a method of manufacturing high-quality epsilon-caprolactam in good yield by crystallizing raw epsilon-caprolactam containing an impurity, that was obtained by Beckmann rearrangement of cyclohexanoneoxime.

### BACKGROUND ART

Epsilon-caprolactam is an important compound as an intermediate for manufacturing nylon-6, and various methods of manufacturing the same have been known. For example, epsilon-caprolactam can be mass-produced by Beckmann rearrangement of cyclohexanoneoxime in the presence of an acid medium such as fuming sulfuric acid. This method gives rise to a problem of a large amount of a by-product, that is, ammonium sulfate low in added values.

As a method of solving the problem, a method of manufacturing epsilon-caprolactam based on vapor phase Beckmann rearrangement reaction with the use of a solid catalyst has been known. As a solid catalyst to be used for vapor phase Beckmann rearrangement reaction, a boric-acid-based catalyst, a silica-alumina catalyst, a solid phosphoric acid catalyst, a composite metal oxide catalyst, a zeolite-based catalyst, and the like have been proposed.

Epsilon-caprolactam obtained with this method, however, contains various impurities. As is well known, though epsilon-caprolactam is used as a raw material for polyamide, epsilon-caprolactam for manufacturing polyamides to be used for synthetic fibers or films is required to be high in purity. Therefore, raw epsilon-caprolactam obtained with the method above is normally purified with various methods such as crystallization, extraction, distillation, hydrogenation, and the like.

Among these purification methods, a crystallization method is more advantageous in terms of energy than a distillation method and the like, and it has been known as a method capable of removing a relatively large amount of impurity at once. As the crystallization method, a cooling crystallization method of precipitating crystal by cooling a liquid to be separated, an evaporative crystallization method of precipitating crystal by evaporating and removing a solvent in a liquid to be separated for condensation, an antisolvent crystallization method of precipitating crystal by adding a poor solvent to a liquid to be separated to thereby lower a degree at which a substance of interest is dissolved, a melt crystallization method of removing impurities by raising a temperature of crystal after the crystal was precipitated with various methods, and the like have frequently been used industrially.

Patent Document 1 discloses a method of manufacturing purified epsilon-caprolactam by pouring molten raw epsilon-caprolactam and a cooled organic solvent together and mixing the same to thereby crystallize epsilon-caprolactam, and then subjecting the resultant substance to solid-liquid separation.

### CITATION LIST

### Patent Literature

PTL 1: Japanese Patent No. 4182273

### SUMMARY OF INVENTION

### Technical Problem

In normally manufacturing purified epsilon-caprolactam with the use of the crystallization method, epsilon-caprolactam is considerably eluted into a crystallization mother liquor that was subjected to solid-liquid separation. In addition, since high purity is required in manufactured epsilon-caprolactam, crystal obtained as a result of solid-liquid separation is generally washed with a large amount of a cleaning liquid and a considerable amount of epsilon-caprolactam is also eluted into this cleaning liquid. Such elution of epsilon-caprolactam disadvantageously brings about manufacturing loss and hence lower yield.

An object of the present invention is to provide an epsilon-caprolactam manufacturing method capable of manufacturing in good yield, high-quality epsilon-caprolactam containing less impurity.

### Solution to Problem

An epsilon-caprolactam manufacturing method according to the present invention is a method of manufacturing epsilon-caprolactam from cyclohexanoneoxime, having: an epsilon-caprolactam purification step of pouring a heated melt of raw epsilon-caprolactam obtained by Beckmann rearrangement of cyclohexanoneoxime together with a cooled solvent into a crystallizer for crystallization and separating a resultant substance into purified epsilon-caprolactam and a crystallization mother liquor through solid-liquid separation; a first-stage epsilon-caprolactam recovery step of separating the crystallization mother liquor into first recovered epsilon-caprolactam and a first recovered mother liquor through evaporative crystallization for crystallizing epsilon-caprolactam in the crystallization mother liquor while evaporating the solvent in the crystallization mother liquor obtained in the epsilon-caprolactam purification step and through successively performed solid-liquid separation; and a second-stage epsilon-caprolactam recovery step of obtaining second recovered purified epsilon-caprolactam by cooling the first recovered mother liquor obtained in the first-stage epsilon-caprolactam recovery step to crystallize epsilon-caprolactam in the first recovered mother liquor, separating the first recovered mother liquor into second recovered epsilon-caprolactam and a second recovered mother liquor through successively performed solid-liquid separation, and raising a temperature to an elution temperature at which a part of obtained second recovered epsilon-caprolactam is molten without obtained second recovered epsilon-caprolactam being completely molten so as to elute impurities in second recovered epsilon-caprolactam together with a molten substance, wherein first recovered epsilon-caprolactam obtained in the first-stage epsilon-caprolactam recovery step is recovered as a raw material for the epsilon-caprolactam purification step, and second recovered purified epsilon-caprolactam obtained in the second-stage epsilon-caprolactam recovery step is recovered as a raw material for the epsilon-caprolactam purification step and/or the first-stage epsilon-caprolactam recovery step.

The epsilon-caprolactam manufacturing method according to the present invention includes the epsilon-caprolactam purification step, the first-stage epsilon-caprolactam recovery step, and the second-stage epsilon-caprolactam recovery step.

In the epsilon-caprolactam purification step, purified epsilon-caprolactam is obtained in such a manner that a heated melt of raw epsilon-caprolactam obtained by Beckmann rearrangement of cyclohexanoneoxime and a cooled solvent are poured together into a crystallizer for crystallization, followed by solid-liquid separation. By applying a direct cooling type crystallization method in which heated and molten raw epsilon-caprolactam and the cooled solvent are poured together into the crystallizer, scaling that occurs at the heat transfer surface of the crystallizer as seen in an indirect cooling type crystallization method can be suppressed.

In the first-stage epsilon-caprolactam recovery step, first recovered epsilon-caprolactam is obtained by evaporating the solvent in the crystallization mother liquor obtained through solid-liquid separation in the preceding epsilon-caprolactam purification step so as to crystallize epsilon-caprolactam, followed by solid-liquid separation. By performing reduced-pressure cooling crystallization utilizing evaporative latent heat of the crystallization mother liquor containing the solvent produced in the epsilon-caprolactam purification step, scaling at the heat transfer surface of the crystallizer as seen in the indirect cooling type is suppressed, and consequently, high-quality first recovered epsilon-caprolactam is obtained in a stable manner. This obtained first recovered epsilon-caprolactam is recycled as a raw material for the epsilon-caprolactam purification step.

In the second-stage epsilon-caprolactam recovery step, second recovered epsilon-caprolactam is obtained by once subjecting the first recovered mother liquor obtained in the preceding first-stage epsilon-caprolactam recovery step to cooling crystallization so as to crystallize epsilon-caprolactam contained in the first recovered mother liquor, followed by solid-liquid separation, and then second recovered purified epsilon-caprolactam is obtained by raising a temperature to an elution temperature at which a part of the obtained epsilon-caprolactam crystal is molten without the obtained epsilon-caprolactam crystal being completely molten so as to elute impurities contained in the crystal and completely melting finally remaining crystal. This obtained second recovered purified epsilon-caprolactam is recycled as a raw material for the epsilon-caprolactam purification step and/or the first-stage epsilon-caprolactam recovery step. Since the melt crystallization method is applied in the second-stage epsilon-caprolactam recovery step, impurities can readily be removed by elution and thus second recovered purified epsilon-caprolactam low in impurity concentration and suitable for the epsilon-caprolactam purification step and/or the first-stage epsilon-caprolactam recovery step is obtained.

According to the epsilon-caprolactam manufacturing method in the present invention, by combining the epsilon-caprolactam purification step, the first-stage epsilon-caprolactam recovery step, and the second-stage epsilon-caprolactam recovery step as above, high-quality purified epsilon-caprolactam less in production loss of epsilon-caprolactam is obtained.

The epsilon-caprolactam manufacturing method according to the present invention is characterized in that raw epsilon-caprolactam is obtained by vapor phase Beckmann rearrangement of cyclohexanoneoxime by using a solid catalyst.

According to the epsilon-caprolactam manufacturing method in the present invention, raw epsilon-caprolactam to serve as a raw material for the epsilon-caprolactam purification step is obtained by removing impurities through distillation or the like from a reaction mixture obtained by vapor phase Beckmann rearrangement of cyclohexanoneoxime by using a solid catalyst.

The epsilon-caprolactam manufacturing method according to the present invention is characterized in that second recovered epsilon-caprolactam is obtained by crystallizing epsilon-caprolactam in the first recovered mother liquor on a cooled wall surface of the crystallizer, and after the second recovered mother liquor is separated through successively performed solid-liquid separation, a temperature of second recovered epsilon-caprolactam crystallized on the wall surface is raised to the elution temperature by raising a temperature of the wall surface.

According to the epsilon-caprolactam manufacturing method in the present invention, epsilon-caprolactam in the first recovered mother liquor is crystallized on the cooled wall surface of the melt crystallizer, the second recovered mother liquor is separated, and thereafter a temperature of the wall surface is raised to an elution temperature at which only a part of crystallized second recovered epsilon-caprolactam is molten. After the temperature is raised to the elution temperature, second recovered epsilon-caprolactam remaining as crystal on the wall surface is recovered as a melt by raising a temperature of the wall surface to a melting point or higher of second recovered epsilon-caprolactam. Therefore, since the crystal on the wall surface is completely molten, scaling growth on the wall surface over time is not observed and stable melt crystallization can be achieved.

### Advantageous Effects of Invention

The epsilon-caprolactam manufacturing method according to the present invention has the epsilon-caprolactam purification step in which a drop crystallization method where antisolvent crystallization and cooling crystallization are combined is applied, the first-stage epsilon-caprolactam recovery step in which the evaporative crystallization method is applied, and the second-stage epsilon-caprolactam recovery step in which the melt crystallization method is applied. Therefore, raw materials can effectively be made use of and high-quality epsilon-caprolactam can be manufactured in good yield. In addition, since the yield is improved, cost for epsilon-caprolactam itself can be reduced.

### BRIEF DESCRIPTION OF DRAWINGS

FIG. 1 is a diagram showing a process step in an epsilon-caprolactam manufacturing method according to the present invention.

FIG. 2 is a diagram showing a laboratory-scale experiment apparatus of melt crystallization.

### DESCRIPTION OF EMBODIMENTS

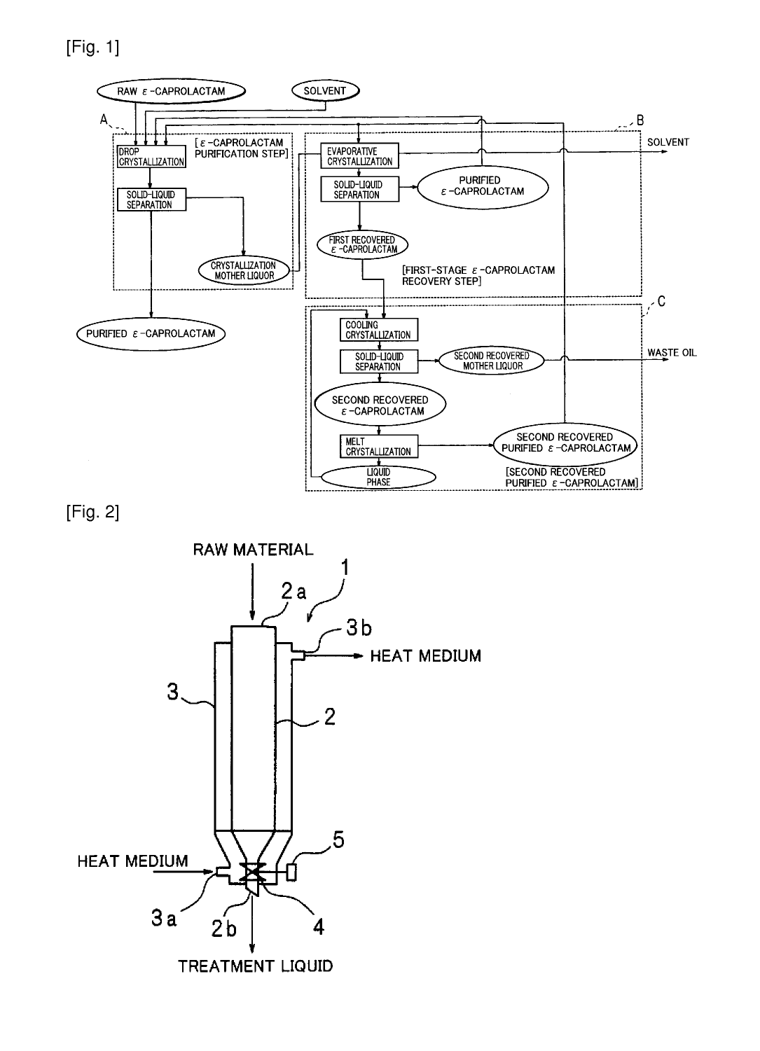

An embodiment of the present invention will be described hereinafter. FIG. 1 is a diagram showing a process step in an epsilon-caprolactam manufacturing method according to the present invention.

The epsilon-caprolactam manufacturing method according to the present invention includes an epsilon-caprolactam purification step A, a first-stage epsilon-caprolactam recovery step B, and a second-stage epsilon-caprolactam recovery step C. Each step will be described hereinafter in detail.

(Epsilon-Caprolactam Purification Step A)

Raw epsilon-caprolactam is obtained by Beckmann vapor phase rearrangement reaction of cyclohexanoneoxime with the use of a solid catalyst. A zeolite-based catalyst is suitably employed as a solid catalyst. Raw epsilon-caprolactam in a state molten by heating is poured together with the cooled solvent into a crystallizer for drop crystallization [drop crystallization]. Straight chain aliphatic hydrocarbon, side chain aliphatic hydrocarbon, alicyclic hydrocarbon, and the like having carbon number from 6 to 12 are exemplified as the solvent. The solvent, such as n-heptane and cyclohexane, which are insufficient solvent for epsilon-caprolactam, is preferred. A crystallization temperature approximately from 40° C. to 60° C. is preferred. An epsilon-caprolactam solution in a slurry state after crystallization treatment is introduced in a solid-liquid separation machine such as a centrifugal decanter, a centrifugal filter, or the like. The introduced slurry solution is separated into a solid phase composed of epsilon-caprolactam crystal and a liquid phase containing impurities [solid-liquid separation]. By washing epsilon-caprolactam crystal with an organic solvent in order to remove the impurities attached to the crystal, higher-purity epsilon-caprolactam can also be obtained.

In the epsilon-caprolactam purification step, heated and molten raw epsilon-caprolactam and the cooled solvent are poured together into the crystallizer, so that scaling at the heat transfer surface as seen in the indirect cooling crystallization method can be suppressed. In addition, increase in cost for facilities for a vacuum bath for pressure reduction or improvement in pressure resistance of a crystallizer that is required in adopting the evaporative crystallization method can be avoided. Moreover, in order to ensure quality and yield of purified epsilon-caprolactam obtained with the melt crystallization method, a liquid produced at the time of elution of impurities should be recovered. Therefore, if the melt crystallization method is applied in the epsilon-caprolactam purification step high in throughput, it is possible that a facility size and the number of apparatuses will be enormous. By applying the drop crystallization method in the epsilon-caprolactam purification step, however, such disadvantages can be avoided.

(First-Stage Epsilon-Caprolactam Recovery Step B)

The crystallization mother liquor obtained in the epsilon-caprolactam purification step A is poured into a crystallizer and the solvent in the crystallization mother liquor is evaporated to crystallize epsilon-caprolactam [evaporative crystallization]. The crystallization temperature approximately from 20° C. to 60° C. is preferred. An epsilon-caprolactam solution in a slurry state after crystallization treatment is introduced in a solid-liquid separation machine. The introduced slurry solution is separated into a solid phase composed of epsilon-caprolactam crystal and a liquid phase containing impurities [solid-liquid separation].

Higher-purity epsilon-caprolactam can also be obtained by washing epsilon-caprolactam crystal with an organic solvent in order to remove impurities attached to the crystal. This obtained first recovered epsilon-caprolactam is recycled as a raw material for the epsilon-caprolactam purification step A. Namely, this first recovered epsilon-caprolactam is poured into the crystallizer in the epsilon-caprolactam purification step A after it is molten.

In the first-stage epsilon-caprolactam recovery step B, the crystallization mother liquor obtained through solid-liquid separation in the epsilon-caprolactam purification step A is evaporated to crystallize epsilon-caprolactam, and first recovered epsilon-caprolactam is obtained through successively performed solid-liquid separation. By carrying out reduced-pressure cooling crystallization utilizing evaporation latent heat of the crystallization mother liquor containing the solvent produced in the epsilon-caprolactam purification step A, scaling at the heat transfer surface of the crystallizer as seen in the indirect cooling type is suppressed, and consequently high-quality recovered epsilon-caprolactam can be obtained in a stable manner.

In the first-stage epsilon-caprolactam recovery step B, as in the epsilon-caprolactam purification step A, though crystallization by adding another cooled solvent to the crystallization mother liquor as it is or to the crystallization mother liquor obtained in the epsilon-caprolactam purification step A from which the added solvent was separated by distillation is possible, cost for facilities for recovering the added solvent and for recovering the solvent is incurred, which is not realistic. On the other hand, in a case of the indirect cooling crystallization type, scaling at the heat transfer surface occurs and continuous stable operations are difficult. Therefore, the technique according to the present invention in which cooling crystallization is performed by utilizing evaporation latent heat of the solvent while evaporating the contained solvent under reduced pressure (the evaporative crystallization method) is most efficient, because it can also suppress scaling.

(Second-Stage Epsilon-Caprolactam Recovery Step C)

The first recovered mother liquor obtained in the first-stage epsilon-caprolactam recovery step B is poured into a melt crystallizer and cooled, so that epsilon-caprolactam in the first recovered mother liquor is crystallized [cooling crystallization]. A solid-liquid mixture resulting from the cooling crystallization is separated into a solid phase containing epsilon-caprolactam crystal (second recovered epsilon-caprolactam) and a liquid phase containing impurities (second recovered mother liquor) [solid-liquid separation]. The separated liquid phase (second recovered mother liquor) is exhausted to the outside as waste oil.

Second recovered purified epsilon-caprolactam is obtained by raising a temperature of separated second recovered epsilon-caprolactam to an elution temperature at which a part thereof is molten without second recovered epsilon-caprolactam being completely molten so as to elute impurities in second recovered epsilon-caprolactam together with a molten substance [melt crystallization]. This eluted liquid is desirably recovered as a raw material for the second-stage epsilon-caprolactam recovery step C in order to enhance a rate of recovery of epsilon-caprolactam, however, it may be exhausted to the outside.

Obtained second recovered purified epsilon-caprolactam is recycled as a raw material for the epsilon-caprolactam purification step A and/or a raw material for the first-stage epsilon-caprolactam recovery step B. Namely, this second recovered purified epsilon-caprolactam is poured into the crystallizer in the epsilon-caprolactam purification step A and/or the crystallizer in the first-stage epsilon-caprolactam recovery step B as a heated melt after it is molten.

In the second-stage epsilon-caprolactam recovery step C, concentration of impurities in the first recovered mother liquor obtained in the first-stage epsilon-caprolactam recovery step B and serving as the raw material is very high. Therefore, when the crystal and the mother liquor are simply subjected to solid-liquid separation by performing normal cooling crystallization, a large amount of impurity remains in the crystal. It is thus difficult to achieve satisfactory crystal quality even though the crystal is washed with a cleaning liquid. In addition, in a case where a raw material containing a large amount of impurity is subjected to cooling crystallization, viscosity of an obtained slurry solution is high, which may lead not only to occurrence of scaling at the heat transfer surface but also clogging by the slurry solution of a pipe or the like for extracting the slurry solution from the crystallizer. Namely, there is a concern about stable continuous operations.

In recovering epsilon-caprolactam, as the number of steps in recovery thereof increases, concentration of impurity in the raw material such as the crystallization mother liquor supplied to each recovery step becomes significantly high and hence concentration of impurity in epsilon-caprolactam recovered in the recovery step also becomes high. If such epsilon-caprolactam high in impurity concentration is returned to upstream for recycle, impurities are accumulated in each recovery step and they affect also quality of purified epsilon-caprolactam obtained in the purification step. Consequently, quality of a final product may be out of spec. Therefore, in a case where several recovery steps are adopted, higher purification performance is required in a further subsequent step.

In the second-stage epsilon-caprolactam recovery step C according to the present invention, the melt crystallization method is applied. Therefore, an amount of contained impurity is extremely small, and second recovered purified epsilon-caprolactam high in quality to such an extent as being free from adverse effect on the epsilon-caprolactam purification step and/or the first-stage epsilon-caprolactam recovery step can be recovered. Thus, recycle of this obtained second recovered purified epsilon-caprolactam will not adversely affect each of the epsilon-caprolactam purification step and/or the first-stage epsilon-caprolactam recovery step.

In addition, in recovering second recovered purified epsilon-caprolactam, it is recovered as a heated melt. Therefore, scaling at the heat transfer surface of the crystallizer is eliminated each time the heated melt is recovered, and a problem of occurrence of scaling does not arise.

In a case where purity of molten second recovered purified epsilon-caprolactam does not satisfy a desired standard (a standard at which recycling as a raw material for the epsilon-caprolactam purification step A and/or a raw material for the first-stage epsilon-caprolactam recovery step B is not adversely affected), a melt of this second recovered purified epsilon-caprolactam is not returned to each preceding step but provided to the additional step which is the same step as the second-stage epsilon-caprolactam recovery step C described above. Second recovered purified epsilon-caprolactam obtained in the additional step is recycled as raw material for the epsilon-caprolactam purification step A and/or a raw material for the first stage epsilon-caprolactam recovery step B. By thus adding the second-stage epsilon-caprolactam recovery step C as necessary, second recovered purified epsilon-caprolactam having purity satisfying a desired standard can reliably be recovered in the preceding step.

As described above, in the epsilon-caprolactam manufacturing method according to the present invention, the epsilon-caprolactam purification step A in which the drop crystallization method is applied, the first-stage epsilon-caprolactam recovery step B in which the evaporative crystallization method is applied, and the second-stage epsilon-caprolactam recovery step C in which the melt crystallization method is applied are combined, a substance recovered in the first-stage epsilon-caprolactam recovery step B is recycled as a raw material for the epsilon-caprolactam purification step A, and a substance recovered in the second-stage epsilon-caprolactam recovery step C is recycled as a raw material for the epsilon-caprolactam purification step A and/or a raw material for the first-stage epsilon-caprolactam recovery step B. Therefore, epsilon-caprolactam small in an amount of contained impurity and high in quality can be manufactured in good yield. In addition, in that case, stable continuous operations are also easily performed.

### Example

A specific example of the present invention will be described hereinafter.

(Epsilon-Caprolactam Purification Step A)

The following operations were continuously performed. A flow rate was shown with weight per unit time. A reaction product was obtained by using a high-silica zeolite catalyst and causing vapor phase Beckmann rearrangement reaction of cyclohexanoneoxime at a temperature condition of 380° C. in the coexistence of methanol. A low-melting substance and a high-melting substance were removed from this reaction product by distillation, to thereby obtain raw epsilon-caprolactam. Quality of obtained raw epsilon-caprolactam was found based on GC (Gas Chromatography) analysis as follows: epsilon-caprolactam of 99.131%; cyclohexanoneoxime (OXM) of 139 ppm; 3-N-methyl-4,5,6,7-tetrahydrobenzimidazole (MTHI) of 398 ppm; and 1,2,3,4,6,7,8,9-octahydrophenazine (OHP) of 430 ppm.

This raw epsilon-caprolactam was molten and its temperature was set to 75° C., and 200 parts of the melt thereof and 400 parts of a solvent of n-heptane/cyclohexane=3 (weight ratio) at 5° C. were continuously added by pouring into the crystallizer of which jacket was held at 56° C. A crystallization temperature was set to 55° C. and an average retention time was approximately 30 minutes. A slurry liquid was sent from the crystallizer at a ratio of 600 parts to a centrifugal decanter (a solid-liquid separation machine) of which temperature was kept, a solid was continuously rinsed with 80 parts solvent identical in composition held at approximately 50° C., and crystal was obtained at a ratio of 150 parts and a separated liquid was obtained at a ratio of 530 parts. The obtained crystal was sampled and subjected to GC analysis. Then, epsilon-caprolactam was 96.33%, n-heptane was 2.06%, cyclohexane was 1.26%, and OXM, MTHI and OHP were not detected. The continuous operations as above were successfully performed in a stable manner for 24 hours or longer.

(First-Stage Epsilon-Caprolactam Recovery Step B)

The following operations were continuously performed. A flow rate was shown with weight per unit time. Composition of the separated liquid (crystallization mother liquor) obtained in the epsilon-caprolactam purification step A was as follows: solvent content of 86.35% and n-heptane/cyclohexane=2.75 (weight ratio). In addition, GC analysis values except for the solvent were as follows: epsilon-caprolactam of 97.69%; OXM of 1220 ppm; MTHI of 451 ppm; and OHP of 849 ppm.

Under such conditions as a pressure of 240 Torr and a temperature of 58.5° C., the solvent was distilled out as vapor from 894 parts of this separated liquid. An amount of the distilled-away solvent was 386 parts. A remaining liquid that had not evaporated was poured into the crystallizer at 90 Torr and at 40.6° C. together with 230 parts liquid phase obtained in solid-liquid separation which will be described later, and the solvent was distilled away while a wall of an apparatus or the like of a vapor phase portion was washed with 100 parts solvent. Thus, crystal of epsilon-caprolactam was precipitated. The distilled-out solvent was cooled and 380 parts solvent were recovered by condensation.

An average retention time in the crystallizer was 74 minutes. Here, 468 parts crystal slurry were continuously extracted and subjected to solid-liquid separation in the centrifugal decanter (solid-liquid separation machine) held at 40° C., to thereby obtain 128 parts crystal. This crystal was molten and its composition was analyzed as follows: solvent content of 8.59% and n-heptane/cyclohexane=3.98 (weight ratio). GC analysis values were as follows: epsilon-caprolactam of 99.68%; OXM of 129 ppm; MTHI of 69 ppm; and OHP of 25 ppm.

Through the operations as above, 116.6 parts of 119.2 parts epsilon-caprolactam contained in the separated liquid (crystallization mother liquor) in the epsilon-caprolactam purification step A could be recovered as crystal. In addition, composition of recovered epsilon-caprolactam was higher in purity and lower in concentration of each impurity than the composition of the raw material in the epsilon-caprolactam purification step A. Therefore, by recycling this recovered epsilon-caprolactam (first recovered epsilon-caprolactam) as a raw material for the epsilon-caprolactam purification step A, yield of high-quality epsilon-caprolactam is increased without adverse effect on manufactured purified epsilon-caprolactam.

(Second-Stage Epsilon-Caprolactam Recovery Step C: First Time)

The following operations were performed in a batch. Weight was shown with weight per 1 batch of crystallization. A solvent phase was removed by decantation from solid-liquid separation in the first epsilon-caprolactam recovery step B, to obtain 275.9 parts crystallization raw material. Composition of the obtained crystallization raw material was as follows: solvent content of 17.00% and n-heptane/cyclohexane=4.34 (weight ratio). In addition, GC analysis values except for the solvent were as follows: epsilon-caprolactam of 72.07%; OXM of 2.31%; MTHI of 5335 ppm; and OHP of 7870 ppm.

This crystallization raw material liquid was supplied on an inner pipe side of the melt crystallizer having a jacketed-pipe construction and a heat medium was fed to an outer pipe side such that a temperature of the supplied raw material was set to 40° C. Thereafter, the temperature of the introduced heat medium was adjusted to precipitate crystal (second recovered epsilon-caprolactam) on a wall surface of the inner pipe at a raw material temperature of 0° C. Successively, after an uncrystallized mother liquor (second recovered mother liquor) was exhausted, a temperature of the heat medium was gradually raised such that a temperature of the crystal attained to 53.5° C. Here, a part of the crystal (second recovered epsilon-caprolactam) was molten without the crystal being completely molten. Then, epsilon-caprolactam containing eluted impurities was exhausted.

Finally, the temperature of the heat medium was raised to 80° C. so as to melt a crystalline substance remaining in the inner pipe, and thus 83.5 parts were recovered as the melt. Scaling in the inside of the jacketed pipe of the melt crystallizer after recovery was not at all observed. The recovered melt was composed of a solvent content of 0.39% and n-heptane alone. In addition, GC analysis values except for the solvent were as follows: epsilon-caprolactam of 96.17%; OXM of 3090 ppm; MTHI of 586 ppm; and OHP of 894 ppm.

The composition of epsilon-caprolactam recovered in the operations above was slightly lower in purity of epsilon-caprolactam and higher in concentration of OXM, MTHI and OHP than the composition of the raw material in the first-stage epsilon-caprolactam recovery step B. Therefore, using recovered epsilon-caprolactam obtained in the operations above as the raw material, crystallization was again performed.

(Second-Stage Epsilon-Caprolactam Recovery Step C: Second Time)

The following operations were performed in a batch. Weight was shown with weight per 1 batch of crystallization. The second-stage epsilon-caprolactam recovery step C for the second time was performed by using as the raw material, 75.0 parts recovered epsilon-caprolactam obtained in the second-stage epsilon-caprolactam recovery step C for the first time described above.

This crystallization raw material liquid was supplied on the inner pipe side of the melt crystallizer and a heat medium was fed to the outer pipe side such that a temperature of the supplied raw material was set to 50° C. Thereafter, the temperature of the introduced heat medium was adjusted to precipitate crystal (second recovered epsilon-caprolactam) at a raw material temperature of 40° C. Successively, after an uncrystallized mother liquor (second recovered mother liquor) was exhausted, a temperature of the heat medium was gradually raised such that a temperature of the crystal attained to 58.9° C. Here, epsilon-caprolactam containing eluted impurities was exhausted.

Finally, the temperature of the heat medium was raised to 80° C. so as to melt a crystalline substance remaining in the inner pipe (second recovered purified epsilon-caprolactam), and thus 50.6 parts were recovered as the melt. Scaling in the inside of the jacketed pipe after recovery was not at all observed. Regarding composition of the recovered melt, the solvent content was not higher than a detection limit. In addition, GC analysis values except for the solvent were as follows: epsilon-caprolactam of 98.40%; OXM of 1237 ppm; MTHI of 232 ppm; and OHP of 335 ppm.

Composition of epsilon-caprolactam recovered in the operations above (second recovered purified epsilon-caprolactam) was higher in purity of epsilon-caprolactam and also equal to or lower in impurity concentration than the composition of the raw material in the first-stage epsilon-caprolactam recovery step B. Therefore, even when epsilon-caprolactam recovered in the operations above (second recovered purified epsilon-caprolactam) is returned to the first-stage epsilon-caprolactam recovery step B for recycle, quality of recovered epsilon-caprolactam obtained in the first-stage epsilon-caprolactam recovery step B tends to improve and quality of purified epsilon-caprolactam in further upstream epsilon-caprolactam purification step A is not adversely affected.

In the example described above, a case where second recovered purified epsilon-caprolactam recovered in the second-stage epsilon-caprolactam recovery step C is returned only to the first-stage epsilon-caprolactam recovery step B has been described. If the composition of second recovered purified epsilon-caprolactam recovered in the second-stage epsilon-caprolactam recovery step C is better than the composition of the raw material in the epsilon-caprolactam purification step A, however, that recovered second recovered purified epsilon-caprolactam can naturally be returned to the epsilon-caprolactam purification step A for recycle.

FIG. 2 is a diagram showing a laboratory-scale experiment apparatus of melt crystallization, that is used in the second-stage epsilon-caprolactam recovery step C. Using the present apparatus, melt crystallization as described above was evaluated. A melt crystallizer 1 is constructed as a jacketed pipe made of SUS, that has cylindrical inner pipe 2 and outer pipe 3. A raw material is poured into inner pipe 2 through an upper inlet port 2a, and a liquid phase after treatment is extracted through a lower exhaust port 2b. With regard to outer pipe 3, a heat medium introduced from a lower inlet 3a is exhausted from an upper outlet 3b. As this heat medium circulates, the raw material within inner pipe 2 is maintained at a desired temperature. A valve 4 is provided in the vicinity of exhaust port 2b of inner pipe 2 and opening and closing of valve 4 is controlled by a control unit 5.

An example where an epsilon-caprolactam recovery step, in which the cooling crystallization is applied without the meltcrystallization, was performed as a step following the first-stage epsilon-caprolactam recovery step (the second-stage epsilon-caprolactam recovery step) will be described as a Comparative Example in the present invention.

### Comparative Example

### Second-Stage Epsilon-Caprolactam Recovery Step (Cooling Crystallization)

The following operations were continuously performed. A flow rate was shown with weight per unit time. Composition of a solid-liquid separated liquid obtained in the first-stage epsilon-caprolactam recovery step B was as follows: solvent content of 45.35% and n-heptane/cyclohexane=7.64 (weight ratio). In addition, GC analysis values except for the solvent were as follows: epsilon-caprolactam of 76.68%; OXM of 4018 ppm; MTHI of 3899 ppm; and OHP of 10017 ppm.

Crystal slurry was obtained by crystallizing 11.3 parts of this solid-liquid separated liquid at a temperature of −0.8° C. in continuous indirect cooling. The obtained crystal slurry was subjected to solid-liquid separation with the use of a centrifugal separator, to thereby obtain 3.44 parts epsilon-caprolactam crystal containing impurities and 7.86 parts mother liquor. Crystal of epsilon-caprolactam was washed with cyclohexane in a double amount, and cyclohexane was removed by again using the centrifugal separator. Composition of the obtained crystal was as follows: solvent content of 0.71% and n-heptane/cyclohexane=0.58 (weight ratio). In addition, GC analysis values except for the solvent were as follows: epsilon-caprolactam of 94.11%; OXM of 970 ppm; MTHI of 1130 ppm; and OHP of 217 ppm. Scaling on an indirect cooling surface was observed during the continuous operations. In addition, a line for extracting the crystal slurry from the crystallizer was frequently clogged and a stable operation was difficult.

The composition of obtained epsilon-caprolactam was lower in purity and also higher in MTHI concentration than the composition of the raw material in the first-stage epsilon-caprolactam recovery step B from which recovery was carried out. Therefore, if recovered epsilon-caprolactam obtained in the operations above is recycled in the first-stage epsilon-caprolactam recovery step, impurity concentration in the raw material in that step becomes high and quality of first recovered epsilon-caprolactam obtained in that step becomes poor. Consequently, quality of purified epsilon-caprolactam in the epsilon-caprolactam purification step also becomes poor and quality specifications of a product may not be satisfied.

Though a case where raw epsilon-caprolactam obtained by vapor phase Beckmann rearrangement with the use of a zeolite-based catalyst is employed has been described in the example described above, raw epsilon-caprolactam to which the present invention is applicable is not limited thereto.

### REFERENCE SIGNS LIST

A epsilon-caprolactam purification step
B first-stage epsilon-caprolactam recovery step
C second-stage epsilon-caprolactam recovery step

1. A method of manufacturing epsilon-caprolactam from cyclohexanoneoxime, comprising:
an epsilon-caprolactam purification step of pouring a heated melt of raw epsilon-caprolactam obtained by Beckmann rearrangement of cyclohexanoneoxime together with a cooled solvent into a crystallizer for crystallization and separating a resultant substance into purified epsilon-caprolactam and a crystallization mother liquor through solid-liquid separation;
a first-stage epsilon-caprolactam recovery step of separating said crystallization mother liquor into first recovered epsilon-caprolactam and a first recovered mother liquor through evaporative crystallization for crystallizing epsilon-caprolactam in the crystallization mother liquor while evaporating the solvent in the crystallization mother liquor obtained in the epsilon-caprolactam purification step and through successively performed solid-liquid separation; and
a second-stage epsilon-caprolactam recovery step of obtaining second recovered purified epsilon-caprolactam by cooling the first recovered mother liquor obtained in the first-stage epsilon-caprolactam recovery step to crystallize epsilon-caprolactam in the first recovered mother liquor, separating said first recovered mother liquor into second recovered epsilon-caprolactam and a second recovered mother liquor through successively performed solid-liquid separation, and raising a temperature to an elution temperature at which a part of obtained second recovered epsilon-caprolactam is molten without obtained second recovered epsilon-caprolactam being completely molten so as to elute impurities in second recovered epsilon-caprolactam together with a molten substance, wherein
the first recovered epsilon-caprolactam obtained in said first-stage epsilon-caprolactam recovery step is recovered as a raw material for said epsilon-caprolactam purification step, and the second recovered purified epsilon-caprolactam obtained in said second-stage epsilon-caprolactam recovery step is recovered as a raw material for said epsilon-caprolactam purification step and/or said first-stage epsilon-caprolactam recovery step.

2. The method of manufacturing epsilon-caprolactam according to claim 1, wherein
said raw epsilon-caprolactam is obtained by vapor phase Beckmann rearrangement of cyclohexanoneoxime by using a solid catalyst.

3. The method of manufacturing epsilon-caprolactam according to claim 1 or 2, wherein
second recovered epsilon-caprolactam is obtained by crystallizing epsilon-caprolactam in said first recovered mother liquor on a cooled wall surface of the crystallizer, and after the second recovered mother liquor is separated through successively performed solid-liquid separation, a temperature of second recovered epsilon-caprolactam crystallized on said wall surface is raised to said elution temperature by raising a temperature of the wall surface.
